(19) United States
(12) Patent Application Publication
(10) Pub. No.: US 2004/0258752 A1
(43) Pub. Date: Dec. 23, 2004

(54) Taste masking pharmaceutical composition and process for its preparation

(76) Inventors: Manoj Kumar Paruthi, Sweifiyeh (JO); Shrikant Bhonsle, Thane (IN); Anandi Krishnan, Vashi (IN)

Correspondence Address: Michael E. Carmen, DILWORTH & BARRESE, LLP, 333 Earle Ovington Blvd., Uniondale, NY, 11553, US

(21) Appl. No.: 10/766,248

(22) Filed: Jan. 27, 2004

(30) Foreign Application Priority Data
Jan. 31, 2003 (IN) 131/MUM/2003

(51) Int. Cl. A61K 9/24

(52) U.S. Cl. 424/472

(57) ABSTRACT

A taste-masking, pharmaceutical composition is provided which comprises (a) a core comprising an active pharmaceutical ingredient having a taste for which masking is desired and (b) a taste-masking film coating layer on the core, the taste-masking film coating layer comprising (i) a film-forming, water insoluble polymer and (ii) a pH dependent, water-insoluble polymer which dissolves at a pH level of about 5 or below.

### CROSS REFERENCE TO RELATED APPLICATIONS

This application claims priority from Indian Provisional Application Number 131/Mum/2003, filed Jan. 31, 2003 and Indian Application Serial Number ______, filed ______, the contents of which are incorporated by reference herein.

### BACKGROUND OF THE INVENTION

1. Field of the Invention

The present invention relates generally to an improved pharmaceutical composition that provides taste masking for orally-administered bitter drugs and processes for preparing the same. More particularly, the present invention relates to a pharmaceutical composition that comprises an active pharmaceutical ingredient core region and a taste masking layer formed from an aqueous suspension for application over the core.

2. Description of the Related Art

Most drugs are preferably formulated as oral dosage forms due to the ease of administration and low cost of development. Because the pediatric or elderly patient populations often have difficulty in swallowing a solid tablet, the preferred oral dosage forms for such patients may be a chewable tablet, dispersible tablet, reconstituted powder suspension or an oral liquid solution. Such formulations allow a greater exposure of the drug to the taste buds resulting in problems with patient compliance due to the highly bitter or unpleasant taste some drugs have when administered. In many cases, the objectionable taste cannot be circumvented by use of flavoring or sweetening agents.

Attempts have been made to mask the highly bitter or unpleasant tasting drugs during administration to a patient in need of such drugs. Present taste masking technology generally uses microencapsulation techniques which rely primarily on polymer coating materials applied from non-aqueous solutions. These techniques, however, require the presence of organic solvents which may generate regulatory and safety issues and are discouraged because an increased awareness of health and environmental hazards. Other taste masking techniques such as, for example, lipid entrapments and complexation with ion exchange resins are extremely complex and capital intensive.

Examples of taste masking techniques are described in U.S. Pat. No. 5,075,114 (Assignee: McNeil-PPC, Inc.; Filed: May 23, 1990; Published: Dec. 24, 1991) which discloses chewable medicament tablets made from coated granules of a medicament wherein the coating on the granules comprises a blend of cellulose acetate and/or cellulose acetate butyrate and hydroxy propyl cellulose and a process for making such tablets. However, formulations made from such compositions may give some release of the bitter tasting drug when it is in contact with liquid in the oral cavity resulting in the patient suffering some bad taste of the bitter drug during administration.

Another example is U.S. Pat. No. 5,082,669 (Assignee: Dainippon Pharmaceutical Co., Ltd.; Filed: Jul. 18, 1990; Published: January 21, 1992) which discloses a rapid-releasing oral particle pharmaceutical composition comprising a core containing at least a drug having a bitter or unpleasant taste and a water-swelling agent and a film layer coating the core. The film layer contains at least ethylcellulose and a water-soluble substance wherein the presence of the water soluble substance makes the film permeable to liquids in the oral cavity resulting in the patient suffering some bad taste of the bitter drug during administration.

Yet another example is U.S. Pat. No. 5,728,403 (Assignee: The Board of Regents of the University of Nebraska; Filed: Oct. 5, 1994; Published: Mar. 17, 1998) which discloses a pharmaceutical coating for taste masking oral drug compositions which includes a combination of triglycerides and a polymer. The triglyceride mixture melts at body temperature and the copolymer causes the coating to dissolve upon reaching the acidic environment of the stomach. However, such coatings may pose problems during storage and also the performance of the coating will be affected from patient to patient due to temperature variations in respective patients.

The examples described above are not satisfactory due to the potential for drug leakage during storage or administration. In addition, the above approaches are complex, time consuming and cost inefficient for a commercial scale.

Accordingly, there exists a need to develop an improved taste masking pharmaceutical composition for bitter and unpalatable active pharmaceutical ingredients (“API”)that can remain stable during storage, allow for the proper release of the bitter tasting API's, and are cost and use efficient. SUMMARY OF THE INVENTION

The present invention therefore relates to an improved pharmaceutical composition that provides taste masking for orally-administered bitter and unpalatable drugs and processes for preparing the same. Accordingly, in one embodiment of the present invention an improved pharmaceutical composition is provided which comprises an active pharmaceutical ingredient containing core region and a taste masking layer formed from an aqueous suspension for application over the core.

Another embodiment of the present invention is to provide an improved pharmaceutical composition which is non-permeable for taste masking purposes. The improved pharmaceutical composition of the present invention provides taste masking for oral pharmaceuticals, and maintains integrity during the brief transit period in the mouth while releasing the medication in the gastric fluid of the stomach.

Yet another embodiment of the present invention is to provide a process for preparing the improved pharmaceutical composition herein.

The present invention also relates to a taste-masking film coating layer comprising (a) a film-forming, water-insoluble polymer; and (b) a pH dependent, water-insoluble polymer which dissolves at a pH level of about 5 or below.

In accordance with yet another embodiment of the present invention, a method for taste-masking an API is provided comprising the steps of: (a) forming a core region comprising an API for which taste-masking is desired and at least one pharmaceutically acceptable excipient; and, (b) coating the core region with a taste masking film coating layer, wherein the layer comprises: (i) a film-forming, water-insoluble polymer; and (ii) a pH dependent, water-insoluble polymer which dissolves at a pH level of about 5 or below.

The expression “solid oral dosage composition” as used herein shall be understood to mean all solid oral dosage forms including powders, tablets, dispersible granules, capsules, caplets, sachets and the like.

The terms “treating” or “treatment” of a state, disorder or condition as used herein shall be understood to mean: (1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein shall be understood to mean the amount of a compound that, when administered to a subject for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein shall be understood to mean providing a therapeutically effective amount of an active ingredient to a particular location within a host means causing a therapeutically effective blood concentration of the active ingredient at the particular location. This can be accomplished by, e.g., topical, local or systemic administration of the active ingredient to the host.

By “pharmaceutically acceptable” is meant those salts and esters which are within the scope of sound medical judgment, suitable for use in contact with the tissues of humans and lower animals without undue toxicity, irritation, allergic response and the like, commensurate with a reasonable benefit/risk ratio, and effective for their intended use. Representative acid addition salts include, but are not limited to, the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarare, succinate, tartrate, ascorbate, glucoheptonate, lactobionate, lauryl sulphate salts and the like. Representative alkali or alkaline earth metal salts include, but are not limited to, the sodium, calcium, potassium and magnesium salts, and the like.

The term “subject” or “a patient” or “a host” as used herein refers to mammalian animals, preferably human.

As used herein the term “antioxidant” is intended to mean an agent which inhibits oxidation and is thus used to prevent the deterioration of preparations by the oxidative process. Such compounds include, by way of example and without limitation, ascorbic acid, ascorbic palmitate, Vitamin E, butylated hydroxyanisole, butylated hydroxytoluelle, hypophosphorous acid, monothioglycerol, propyl gallate, sodium ascorbate, sodium bisulfite, sodium formaldehyde sulfoxylate, sodium metalbisultfite and other such materials known to those of ordinary skill in the art.

As used herein, the term “buffering agent” is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

As used herein, the term “binders” is intended to mean substances used to cause adhesion of powder particles in tablet granulations. Such compounds include, by way of example and without limitation, acacia alginic acid, tragacanth, carboxymethylcellulose soduim, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include, but are not limited to, starch, poly(ethylene glycol), guar gum, polysaccharide, bentonites, sugars, invert sugars, poloxamers (PLURONIC™ F68, PLURONIC™ f127), collagen, albumin, celluloses in nonaqueous solvents, combinations therof and the like. Other binders include, for example, poly(propylene glycol), pollyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art

As used herein, the term “diluent” or “filler” is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

As used herein, the term “glidant” is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

As used herein, the terms “lubricant” or “lubricating agent” is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

As used herein, the term “disintegrant” is intended to mean a compound used in solid dosage forms to promote the disruption of the solid mass into smaller particles which are more readily dispersed or dissolved. Exemplary disintegrants include, by way of example and without limitation, starches, e.g., corn starch, potato starch, pre-gelatinixed and modified starched thereof; sweeteners, clays, e.g., bentonite, microcrystalline cellulose (e.g. Avicel™), carsium (e.g. Amberlite™), alginates, sodium starch glycolate, gums, e.g., agar, guar, locust bean, karaya, pectin, tragacanth, combinations thereof and othel Such materials known to those of ordinary skill in the art.

As used herein, the term “wetting agent” is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium steartate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxethylene sorbitan fatty acid esters, (e.g., TWEEN™s), polyethylene glycols, polyoxyethylene stearates colloidal silicon dioxide, phosphates, sodium dodecylsulfate, carboxmethylcellulose calcium, carboxmethylcellulose sodium, methylcellulose, hydroxethylcellulose, hydroxyl propylcellulose, hydroxypropylmethylcellulose phthalate, noncrystalline cellulose, magnesium aluminum silicate, tricthanolamine, polyvinyl alcohol, and polyvinylpryrrolicdone (PVP). Tyloxapol (a nonionic liquid polymet of the alkyl aryl polyether alcohol type, also known as superinone or triton) is another useful wetting agent, combinations thereof and other such materials known to those of ordinary skill in the art.

Most of these excipients are described in detail in Howard C. Ansel et al., Pharmaceutical Dosage Forms and Drug Delivery Systems, 7th Ed. (1999); Alfonso R. Gennaro et al., Remington: The Science and Practice of Pharmacy, 20th Ed. (2000); and A. Kibbe, Handbook of Pharmaceutical Excipients, 3^{rd} Ed. (2000), the contents of which are incorporated by reference herein.

### DETAILED DESCRIPTION OF THE PREFERRED EMBODIMENTS

In accordance with the present invention, improved taste-masking pharmaceutical compositions are provided.

Generally, the compositions of the present invention provide for an improved taste-masking pharmaceutical composition comprising: (a) a core region comprising an API for which taste-masking is desired and at least one pharmaceutically acceptable excipient; and (b) a taste masking film coating layer comprising: (i) a film-forming, water-insoluble ploymer; and (ii) a pH dependent, water-insoluble polymer which dissolves at a pH level of about 5 or below.

In accordance with the present invention, the core of the composition includes at least one or more API's having an unpleasant taste for which taste masking is desired. The “unpleasant taste” is such that when the drug is administered orally, a patient will experience an unpleasant bitter, astringent or irritating sensation.

Representative API's for use in the composition of the present invention include, but are not limited to, antibiotics, analgesics, antihistamines, decongestants, antitussives, steroids, antibacterials, antiepileptics, psychotropics, cardioitonics, antipyretics, antiulcer agents, anti-inflammatories, anti-allergic agents and the like and combinations thereof. A preferred API of the present invention is desloratadine. It is within the scope of the invention, however, to utilize any API that requires taste-masking as set forth herein.

Additional representative API's of the present invention include, but are not limited to, agents used to treat AIDS, e.g., protease inhibitors such as indinavir, ritonavir and saquinavir, and nucleoside analogs such as didanosine, lamivudine, zidovudine and stavudine; heart agents, e.g., captopril, procainamide, labetalol HCl, captopril, diltiazem HCl, enalapril maleate, hydrochlorothiazide, propranolol HCl, mexiletine HCl, procainamide HCl and propafenone HCl; anti-depressants, e.g., clomipramine HCl, desipramine HCl, doxepin HCl and imipramine HCl; antibacterial agents, e.g., metronidazole; antipyretic analgesic anti-inflammatory agents, e.g., acetaminophen, ibuprofen, and flufenamic acid; nootropic agents, e.g., meclofenoxate hydrochloride; antibiotics, e.g., chloramphenicol, josamycin, and erythromycin; antitussives, e.g., aminophylline, and theophylline; central nervous system agents, e.g., indeloxazine hydrochloride and calcium homopantothenate; hypnotic sedatives, e.g., phenobarbital; agents for peptic ulcers, stomachics, antacids, laxatives, cholagogues, and gastrointestinal drugs, e.g., cimetidine, ranitidine, famotidine and calcium carbonate; sympathomimetics, e.g., etilefrine hydrochloride; vasodilators, including diltiazem hydrochloride; β-blockers, e.g., propranolol hydrochloride; drugs for arrhythmia, e.g., digitoxin; antihistamines, e.g., promethazine hydrochloride; anti-malarial agents, e.g., quinine hydrochloride; non-steroidal anti-inflammatory agents, e.g., sulpyrin; expectorants, e.g., ambroxol; as well as other sleep-inducing agents, anti-anxiety drugs, anti-epilepsy drugs, anti-Parkinson's drugs, psychoneurotic drugs, local anesthetics, skeletal muscle relaxants, autonomic nerve drugs, antispasmotics, anti-vertigo drugs, cardiotonics, diuretics, hypotensives, vasoconstrictors, drugs for the circulatory system, drugs for hyperlipidemia, drugs to promote respiration, antitussive expectorants, bronchodilators, antidiarrheal agents, drugs for controlling intestinal function, adrenocortical hormones, hormones, urogenital drugs, vitamins, hemostatics, drugs for liver disease, drugs used for gout, drugs used for diabetes, antibacterials, drugs used against malignant tumors, chemotherapeutic drugs, multisymptom cold medications, nutrition-enhancing health drugs, and osteoporosis drugs. Descriptions of these API's and their adverse taste sensations are described in, for example, Bad Taste of Drugs for AIDS, Heart Disease and Depression can Impair Compliance, Compromise Health, DukeMed News Office, http://dukemednews.duke.edu/news/article.php?id=203, Mar. 28, 2000; Uncommon Solutions to Problems Dosing a Common Drug: Metronidazole, IPS Inc. Prescription Log, http://www.islandpharmacy.com/metronidazole.html, and U.S. Pat. No. 6,656,492, to Kajiyama, et al.

In another embodiment of the present invention, the amount of API requiring taste-masking generally varies from about 1 percent by weight to about 40 percent by weight, based on the total weight of the core of the composition. Preferably, the amount of API ranges varies from about 5 percent by weight to about 35 percent by weight of the core. More preferably, the amount of API varies from about 10 percent by weight to about 30 percent by weight of the core. The core can be prepared by methods generally known in the art used to prepare, for example, ordinary fine granules.

In yet another embodiment of the present invention, the core of the composition can contain various pharmaceutical additives in combination with the API requiring taste-masking. Examples of such additives include, but are not limited to, antioxidants, buffering agents, sweetening agents, binders, diluents, fillers, glidants, lubricating agents, disintegrants, wetting agents and the like and mixtures thereof. Each of the foregoing additives, when used, is used at a functionally effective amount to impart the desired properties to the pharmaceutical formulations herein.

In one embodiment of the present invention, the content of the core in the composition of the present invention generally varies from about 70 percent by weight to about 95 percent by weight, based on the total weight of the composition. Preferably, the core varies from about 80 percent by weight to about 90 percent by weight of tile total coated composition. The shape and size of the core is not limited and may range from truly spherical to irregular and non-uniform. Preferably, the core is finished to granules having a size ranging from about 150 to about 500 microns.

Representative film forming, water insoluble polymers for use in the taste-masking film coating layer of the composition of the present invention include, but are not limited to, ethylcellulose, e.g., Ethocel™ available from Dow Chemical Corp., aqueous polymeric dispersions such as Aquacoat™ (an about 30% w/w aqueous dispersion containing ethyl cellulose, sodium lauryl sulfate, cetyl alcohol and hydrogen peroxide with a pH of about 4.0-7.0) available from FMC, and Surelease™ (a plasticized 25% w/w aqueous dispersion containing ethyl cellulose, ammonium hydroxide, medium chain triglycerides & oleic acid with a pH of about 9.5-11.5) available from Colorcon, polyvinyl acetate, cellulose acetate butyrate, and copolymers of polymethacrylic acid available from Rohm Pharma GmbH under the tradename Eudragit™ (e.g., Eudragit L30D-55, Eudragit L100-55, Eudragit RS30D and Eudragit RL30D). Most preferably, the film forming, water insoluble polymer of the present invention is ethylcellulose.

The pH dependent, water insoluble polymer of the for use in the taste-masking film coating layer advantageously dissolves under acidic conditions of the stomach (e.g., gastric juices), e.g., at a pH level of about 5 or below. Preferably, the pH dependent polymer of the present invention is a polymetliacrylic acid copolymer. A preferred polymethacrylic acid polymer is available from Rohm Pharma GmbH under the tradename Eudragit™ (e.g., Eudragit L30D-55, Eudragit L100-55, Eudragit RS30D and Eudragit RL30D). Most preferably, the pH dependent copolymer of the present invention is Eudragit EPO (Rohm Pharma). The pH dependent polymer is present as discrete particles in the coating.

According to the present invention, once a patient places the composition in the mouth, the coating layer substantially maintains its integrity during the brief transit period in the mouth. The coating layer remains intact because the pH dependent polymer will only dissolve once it is exposed to acidic conditions of the stomach, i.e., at a pH of about 5 or below which is much more acidic than the pH of the mouth. Once the composition enters the acidic environment of the stomach, dissolution occurs and the medication is then available for absorption by the body.

In a preferred embodiment, the coating layer will generally be formed from an aqueous mixture or dispersion comprising: (a) a film-forming water insoluble polymer in an amount varying from about 25 percent by weight to about 75 percent by weight of the total weight of the coating layer; and (b) a pH dependent, water insoluble polymer in an amount varying from about 30 percent by weight to about 70 percent by weight of the total weight of the coating layer. Preferably, the film forming water insoluble polymer will be present in an amount varying from about 40 percent by weight to about 60 percent by weight of the total weight of the coating layer and the pH dependent polymer is present in an amount varying from about 35 percent by weight to about 55 percent by weight of the total weight of the coating layer.

In a particularly preferred embodiment of this invention the film-forming, water insoluble polymer is ethylcellulose and the pH dependent water insoluble polymer is Eudragit EPO. Generally, the taste-masking pharmaceutical composition is prepared by mixing the film-forming water insoluble polymers and pH dependent, water insoluble polymer in a weight ratio of about 1:1, and spraying onto the pharmaceutical cores containing at least one or more API's, e.g., a desloratadine API, and at least one other pharmaceutically acceptable excipient, including, e.g., fillers, lubricants and binders.

The coating layer of the present invention can be applied to the cores described hereinabove by conventional techniques. For example, spray techniques such as top spray, bottom spray and tangential spray techniques using, for example, a fluidized bed coater. For example, air (which may be heated) passes through a bed of the active ingredient granules to fluidize them, and the aqueous solution of the two water insoluble polymers is sprayed onto the fluidized bed and thereby coats the granules. The air passing through the bed dries the coated granules, so that a dry coated granule is obtained. The coated granules can then be used in combination with various excipients, flavors, and colors to make a taste-masking film coated solid oral dosage composition of the present invention.

The dried coating as applied generally varies from about 5 percent by weight to about 30 percent by weight of the total dry weight of the coated composition. Preferably, the coating varies from about 10 percent by weight to about 20 percent by weight of the total dry weight of the coated composition. The exact proportions of coating to the core desired for individual cases can be determined by routine experimentation. The amount of coating may be varied in light of the intended application and desired bulk of the products.

The following examples are provided to enable one skilled in the art to practice the invention and are merely illustrative of the invention. The examples should not be read as limiting the scope of the invention as defined in the claims.

### EXAMPLE

### I. Preparation of Core Granules

| Ingredient | Amount (mg) |
|---|---|
| Granulation | |
| Desloratadine | 5.00 |
| Lactose anhydrous | 13.00 |
| Microcrystalline Cellulose | 19.50 |
| Calcium Carboxymethyl cellulose | 3.25 |
| Low substituted Hydroxy Propylcellulose | 2.20 |
| Lubrication | |
| Colloidal Silicon dioxide | 0.10 |
| Magnesium Stearate | 0.45 |

The intragranular materials were blended and granulated after lubrication. The granules obtained were sized to a suitable size range 150 to 500 microns to provide a core for coating.

### II. Taste Masking Film Coating of Core Granules

| Ingredient | Amount (mg) |
|---|---|
| Ethyl cellulose aqueous dispersion | 14.5 |
| Eudragit EPO | 2.75 |
| Purified Water | q.s |

A taste masking film coating dispersion was prepared by suspending Eudragit EPO powder in water and homogenized. The resulting suspension was then added to an ethylcellulose aqueous dispersion under stirring to provide an aqueous coating dispersion.

Next, the core prepared above, in granular form, is placed in a fluidized bed coater and is fluidized by a flow of warm air. The temperature of the air is not narrowly critical, and can vary over a wide range, keeping in mind the fact that the temperature should not be high enough to cause decomposition, sintering, or melting of tile active ingredient granules. When coating desloratadine granules, a temperature of from about 60° C. to about 80° C. is suitable but such temperature ranges will change depending on the specific active ingredient being coated. The rate of air flow is adjusted so as to fluidize the granules. Such flow will vary depending on factors such as the specific equipment used, the size of the charge of granules, the size of the individual granules, the apparent specific gravity of the granules, and other factors that are known to the worker in the arts relating to fluidized bed coating.

After the core has been fluidized, the aqueous coating dispersion was then sprayed over the fluidized uncoated core granules using a fluidized bed coater with bottom spray attachment (Glatt GPCG1). After the optimal weight gain, the coated granules were dried to an optimal moisture level, mixed with suitable pharmaceutical excipients and compressed by standard procedures to provide a desirable tablet dosage form.

### III. Dissolution Test

In accordance with Pharmacopoeial method U.S.P. apparatus 2 described in <711>U.S.P. 27, p. 2303, the coated particles prepared above were subjected to dissolution in 0.1 N HCl media and it was surprisingly found that a substantial amount of desloratadine was released in the first 5 minutes and the complete release took place within 15 minutes.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. A taste-masking pharmaceutical composition comprising:
(a) a core region comprising an active pharmaceutical ingredient for which taste-masking is desired and at least one pharmaceutically acceptable excipient; and,
(b) a taste-masking film coating layer over the core, the taste-masking film coating layer comprising:
(i) a film-forming, water-insoluble polymer; and,
(ii) a pH dependent, water-insoluble polymer which dissolves at a pH level of about 5 or below.

2. The composition of claim 1, wherein the active pharmaceutical ingredient is desloratadine or it's pharmaceutically acceptable hydrates, salts or esters.

3. The composition of claim 1, wherein the film-forming, water-insoluble polymer is selected from the group consisting of ethylcellulose, polyvinyl acetate, cellulose acetate butyrate, methacrylic acid copolymers and mixtures thereof.

4. The composition of claim 1, wherein the pH dependent, water-insoluble polymer is a copolymer.

5. The composition of claim 3, wherein the copolymer is a polymethacrylic acid copolymer.

6. The composition of claim 1, wherein the film-forming, water-insoluble polymer is ethylcellulose and the pH dependent, water-insoluble polymer is a polymethacrylic acid copolymer.

7. The composition of claim 1, wherein the film-forming, water insoluble polymer is present in an amount from about 25 wt. % to about 75 wt. % and the pH dependent, water-insoluble polymer is present in an amount from about 30 wt. % to about 70 wt. %, based on the total weight of the coating layer.

8. The composition of claim 1, wherein the film forming, water insoluble polymer is present in an amount from about 40 wt. % to about 60 wt. % and the pH dependent, water-insoluble polymer is present in an amount from about 35 wt. % to about 55 wt. %, based on the total weight of the coating layer.

9. The composition of claim 1, wherein the pH dependent, water-insoluble polymer is present as discrete particles distributed homogeneously throughout the coating layer.

10. The composition of claim 1, wherein the active pharmaceutical ingredient is selected from the group consisting of antibiotics, analgesics, antihistamines, decongestants, antitussives, steroids, antibacterials, antiepileptics, psychotropics, cardioitonics, antipyretics, antiulcer agents, anti-inflammatories, anti-allergic agents and mixtures thereof.

11. The composition of claim 1, wherein the amount of the active pharmaceutical ingredient in the core is from about 1 wt. % to about 40 wt. %, based on the total weight of the core.

12. The composition of claim 2, wherein the amount of the active pharmaceutical ingredient in the core is from about 1 wt. % to about 40 wt. %, based on the total weight of the core.

13. The composition of claim 1, wherein the amount of the active pharmaceutical ingredient in the core is from about 5 wt. % to about 35 wt. %, based on the total weight of the core.

14. The composition of claim 2, wherein the amount of the active pharmaceutical ingredient in the core is from about 5 wt. % to about 35 wt. %, based on the total weight of the core.

15. The composition of claim 1, wherein the amount of the active pharmaceutical ingredient in the core is from about 10 wt. % to about 30 wt. %, based on the total weight of the core.

16. The composition of claim 1, further comprising one or more pharmaceutical additives.

17. The composition of claim 14, wherein the one or more pharmaceutical additives is selected from the group consisting antioxidants, buffering agents, sweetening agents, binders, diluents, fillers, glidants, lubricating agents, disintegrants, and wetting agents.

18. The composition of claim 1, wherein the core is present in an amount of about 70 wt. % to about 95 wt. % and the coating is present in an amount of about 5 wt. % to about wt. 30 wt. %, based on the weight of the composition.

19. The composition of claim 1, wherein the core is present in an amount of about 80 wt. % to about 90 wt. % and the coating is present in an amount of about 10 wt. % to about 20 wt. %, based on the weight of the composition.

20. A process for preparing a taste-masking, film coated solid oral dosage composition comprising the step of coating a core comprising an active pharmaceutical ingredient having a taste for which masking is desired with a taste masking, film coating layer the taste-masking, film coating layer comprising (i) a film-forming, water-insoluble polymer; and, (ii) a pH dependent, water-insoluble polymer which dissolves at a pH level of about 5 or below.

21. The process of claim 20, wherein the active pharmaceutical ingredient is selected from the group consisting of antibiotics, analgesics, antihistamines, decongestants, antitussives, steroids, antibacterials, antiepileptics, psychotropics, cardioitonics, antipyretics, antiulcer agents, anti-inflammatories, anti-allergic agents and mixtures thereof.

22. The process of claim 20, wherein the active pharmaceutical ingredient is desloratadine or it's pharmaceutically acceptable hydrates, salts or esters.

23. The process of claim 20, wherein the amount of the active pharmaceutical ingredient in the core is from about 1 wt. % to about 40 wt. %, based on the total weight of the core.

24. The process of claim 20, wherein the amount of the active pharmaceutical ingredient in the core is from about 5 wt. % to about 35 wt. %, based on the total weight of the core.

25. The process of claim 20, wherein the amount of the active pharmaceutical ingredient in the core is from about 10 wt. % to about 30 wt. %, based on the total weight of the core.

26. The process of claim 20, wherein the composition further comprises one or more pharmaceutical additives.

27. The process of claim 26, wherein the one or more pharmaceutical additives is selected from the group consisting of binders, disintegrants, wetting agents, diluents, lubricating agents, and fillers.

28. The process of claim 20 wherein the core is present in an amount of about 75 wt. % to about 95 wt. %, based on the weight of the composition.

29. The process of claim 20, wherein the core is present in an amount of about 80 wt. % to about 90 wt. %, based on the weight of the composition.

30. The process of claim 20, wherein the pH dependent, water-insoluble polymer is present as discrete particles distributed homogeneously throughout the coating layer.

31. The process of claim 20, wherein the film-forming, water-insoluble polymer is selected from the group consisting of ethylcellulose, polyvinyl acetate, cellulose acetate butyrate, copolymers of acrylic acid and mixtures thereof.

32. The process of claim 20, wherein the pH dependent, water-insoluble polymer is a copolymer.

33. The process of claim 32, wherein the copolymer is a polymethacrylic acid copolymer.

34. The process of claim 20, wherein the film-forming, water-insoluble polymer is ethylcellulose and the pH dependent, water-insoluble polymer is a polymethacrylic acid copolymer.

35. The process of claim 20, wherein the film-forming, water insoluble polymer is present in an amount from about 25 wt. % to about 75 wt. % and the pH dependent, water-insoluble polymer is present in an amount from about 30 wt. % to about 70 wt. %, based on the total weight of the coating layer.

36. The process of claim 20, wherein the film forming, water insoluble polymer is present in an amount from about 40 wt. % to about 60 wt. % and the pH dependent, water-insoluble polymer is present in an amount from about 35 wt. % to about 55 wt. %, based on the total weight of the coating layer.

37. The process of claim 34, wherein the pH dependent, water-insoluble polymer is present as discrete particles distributed homogeneously throughout the coating layer.
